(19) United States
(12) Patent Application Publication
(10) Pub. No.: US 2010/0021549 A1
(43) Pub. Date: Jan. 28, 2010

(54) Microparticle oral form useful for the modified release of nanoparticles

(75) Inventors: Rémi Meyrueix, Lyon (FR); Rafael Jorda, Merignac (FR); Anne-Sophie Daviaud, Saint Genis Laval (FR); Alain Constancis, Lyon (FR)

Correspondence Address: PATTON BOGGS LLP, 8484 WESTPARK DRIVE, SUITE 900, MCLEAN, VA, 22102, US

(73) Assignee: Flamel Technologies, S.A., Vénissieux Cedex (FR)

(21) Appl. No.: 12/509,109

(22) Filed: Jul. 24, 2009

Related U.S. Application Data
(60) Provisional application No. 61/084,104, filed on Jul. 28, 2008.

(30) Foreign Application Priority Data
Jul. 28, 2008 (FR) FR 08 55179
Jul. 24, 2009 (FR) PCT/FR2009/051495

(51) Int. Cl. A61K 9/14; A61K 38/02; A61K 38/28; A61K 8/73; A61K 8/64; A61K 31/715; A61K 31/7088

(52) U.S. Cl. 424/492; 424/490; 424/491; 424/493; 424/494; 424/497; 514/2; 514/3; 514/8; 514/54; 977/773; 977/906

(57) ABSTRACT

The present invention aims to propose novel microparticle oral forms for the modified release of active ingredient(s), in particular protein or peptide in nature. It also relates to the uses, in particular therapeutic or cosmetic, of these microparticle oral forms.

The present invention aims to propose novel microparticle oral forms for the modified release of active ingredient(s), abbreviated to “AI” in particular protein or peptide in nature. It also relates to the uses, in particular therapeutic or cosmetic, of these microparticle oral forms.

Among all of the administration methods considered for active ingredients whether these are therapeutic, prophylactic or cosmetic, the oral route is particularly popular, in particular in view of its comfort for the patient and its compatibility with a wide variety of formulations.

Unfortunately, this administration method, which exposes the ingested active ingredient, during its travel through the gastrointestinal tract, under very varied physiological conditions in particular as a function of the pH can, with regard to certain active ingredients, cause problems of bioavailability, due for example to the degradation of the active ingredient in an acid medium. Moreover, it is imperative with regard to certain active ingredients to guarantee a specific release process according to the location of their absorption window.

Multiparticle oral dosage forms have already been developed in order to be satisfactory in this respect.

These multiparticle forms are generally in the form of microparticles or microcapsules the core of which, containing the active ingredient or a mixture of active ingredients, is covered with a coating the composition and/or the thickness of which are precisely adjusted in order to control the release of this active ingredient.

These microparticle systems constituted by a plurality of microcapsules with a diameter generally less than 2000 μm thus prove particularly effective for guaranteeing a delayed and controlled release.

By way of illustration of these controlled release forms in the multiparticle state, there can in particular be mentioned those described in the documents US 2002/0192285, U.S. Pat. No. 6,238,703, US 2002/0192285, US 2005/0118268 and U.S. Pat. No. 5,800,836 and quite particularly those described in the Application WO 03/030878. The document WO 03/03878 proposes a multiparticle system for the oral administration of at least one active ingredient the release of which is controlled over time and as a function of the pH via the chemical nature of the envelope covering the core of the microparticles which contains the active ingredient. More precisely, this envelope is formed by a material comprising at least one hydrophilic polymer bearing ionized groups at neutral pH, such as for example a (meth)acrylic acid and alkyl (meth)acrylate copolymer and at least one hydrophobic compound such as a hydrogenated vegetable wax.

These microparticle systems which are particularly useful for reliably controlling, on the one hand, the transport of the active ingredient that they convey through the gastro-intestinal tract and, on the other hand, the release thereof in the small intestine or if appropriate in the stomach for example, unfortunately prove not to be appropriate for the transport of active ingredients which exhibit reduced stability and/or absorption. This lack of stability can be the consequence of too-rapid degradation due to exposure to an aggressive environment such as the gastro-intestinal lumen which has a very acid pH and/or contains enzymes which act on these active ingredients. As regards the reduced absorption, this can also be due to very low solubility or also to insufficient permeability of the epithelial membrane vis-à-vis the active ingredient considered.

A particular subject of the present invention is to propose a novel microparticle system by oral route aimed at resolving these problems and therefore particularly useful for the targeting of active ingredients such as proteins, glycoproteins, peptides, polysaccharides, lipopolysaccharides, oligo- or polynucleotides as well as small molecules, in particular hydrophobic molecules.

More precisely, an aspect of the present invention is to propose an oral form constituted mainly of reservoir-type microparticles releasing in controlled manner an active ingredient, itself non-covalently combined, at least in part, with nanoparticles of at least one polymer, abbreviated to “POM”. This system differs from the conventional reservoir-type microparticle systems which release the active ingredient that they contain in a non-combined form.

Thus, the present invention, according to a first of its aspects, relates to a microparticle oral form, useful for conditioning at least one active ingredient and releasing in vivo this active ingredient according to a release profile controlled as a function of the pH and/or of time, comprising at least microparticles having a core containing at least said active ingredient and coated with at least one coating layer influencing said release profile of said active ingredient characterized in that

the coating layer is formed from a material comprising at least one polymer A having a solubilization pH value within the pH range from 5 to 7 combined with at least one hydrophobic compound B, and

said active ingredient, present in said core of the microparticles, is at least in part non-covalently combined with nanoparticles formed from at least one polymer POM comprising a hydrophilic hydrocarbon chain bearing one or more hydrophobic groups (G) or an amphiphilic hydrocarbon chain.

Within the meaning of the invention, by the term “conditioning” is meant the ability of the microparticles according to the invention to contain and convey said active ingredient.

According to another of its aspects, the invention also relates to a method for the preparation of microparticles useful for conditioning at least one active ingredient and releasing in vivo this active ingredient according to a release profile controlled as a function of the pH and/or of time, said microparticles having a core containing at least said active ingredient and coated with at least one coating layer influencing said release profile of said active ingredient, said method comprising at least the stages consisting of:

a) having at least one active ingredient non-covalently combined with nanoparticles formed from at least one polymer POM comprising a hydrophilic hydrocarbon chain bearing one or more hydrophobic groups (G) or comprising an amphiphilic hydrocarbon chain,

b) forming from the nanoparticles of stage a) a core comprising said nanoparticles and one or more excipients,

c) forming, from at least one polymer A having a solubilization pH value within the pH range from 5 to 7 and at least one hydrophobic compound B, a coating layer arranged around the core formed in stage b), and

d) recovering the expected microparticles.

Stage b) can be carried out using any conventional granulation technique, such as wet granulation, agglomeration, extrusion/spheronization, compacting, atomization or also spray coating.

As regards stage c), this is carried out by any conventional coating technique. It can advantageously be carried out by spraying in a fluidized bed the nanoparticles of stage a) at least one polymer A having a solubilization pH value within the pH range from 5 to 7 combined with at least one hydrophobic compound B.

The present invention results more particularly from the observation by the inventors that incorporation of an active ingredient in a form combined with nanoparticles of at least one polymer POM according to the invention in a controlled-release multiparticle system as defined previously is achievable and that it proves possible to release this combined AI/POM form at its absorption site, generally the intestine, with increased bioavailability and/or duration of absorption in the intestine. Without wishing to be bound by the theory, it can be assumed that after their release from the microparticles, the nanoparticles loaded with active ingredient can, due to their submicronic size, interact with the mucus of the intestine and improve the absorption of the active ingredient which is then progressively released.

As is clear from the examples hereafter, the particulate oral form according to the invention advantageously makes it possible to envisage a release of the active ingredient that it contains according to a sequential mode. In a first phase, the active ingredient, administered by oral route, is released in a form combined with nanoparticles of a polymer POM, this form having increased bioavailability and/or absorption duration compared with the free form of the same active ingredient. It is only in a second phase that the fraction combined with this active ingredient is dissociated from the nanoparticles of polymer POM.

The use of nanoparticles of polymer as considered according to the invention for administration of the active ingredients by parenteral route is known. Thus Flamel Technologies have described a pharmaceutical form in which a therapeutic protein is combined with nanoparticles of a copolyamino acid comprising hydrophobic groups and hydrophilic groups. (WO96/29991; WO 03/04303). The document WO 03/04303 discloses more particularly a polymer of polyamino acid type comprising aspartic residues and/or glutamic residues, with at least some of these residues bearing grafts comprising at least one alpha-tocopherol unit, e.g. polyglutamate or polyaspartate grafted with alpha-tocopherol. These “hydrophobic modified” homopolyamino acids spontaneously form in water a colloidal suspension of nanoparticles which, in an aqueous suspension at pH 7.4, are able to combine easily with at least one active protein. The application PCT/EP2008/055507 for its part proposes biodegradable polyamino acids, which can be converted to colloidal targeting nano- or micro-particles which are able to combine reversibly with active ingredients. These are more particularly amphiphilic copolyglutamates comprising both positive charges at a pH which is neutral or close to neutrality and pendant hydrophobic groups.

However, all of these systems do not make it possible to either adjust a release profile as a function of time and/or of the pH of the active ingredient that they convey, or to protect this active ingredient from the gastric juices and consequently do not prove appropriate for administration by oral route.

The particulate oral forms according to the invention therefore prove to be particularly advantageous for several reasons with regard to conventional particulate systems.

They convey the active ingredient effectively as far as the intended absorption site. They effectively protect the active ingredient that they release at the absorption site from hydrolysis-type degradation or enzymatic digestion for example, which would be directly prejudicial to the demonstration of the sought biological activity through the oral administration of this active ingredient. Finally, they make it possible to effectively control the release profile of the active ingredient that they contain. Thus, in the case of an AI with a wide absorption window, the microparticles can release the AI/POM nanoparticles over a period of less than 12 hours, preferably less than 6 hours or even less than 2 hours. Whereas in the case of an AI with a narrow absorption window, it is essential that the microparticles release, in the intestinal tract, the nanoparticles loaded with active ingredient AI/POM over a short period for example less than 2 hours or better still less than 1 hour. This requirement for release of the nanoparticles over a controlled period is particularly difficult to satisfy for nanoparticles formed from a polymer POM which remains in the acid medium of the stomach during the gastric retention time. It is to the applicant's credit to have identified a family of compositions for coating the microparticles which make it possible to modulate nanoparticles within a very wide range, the release time of the nanoparticles after their passage through an acid medium such as the stomach. Advantageously, the nanoparticles prove not to be affected by a prolonged residence time in the acid medium, and moreover, their individualization is preserved there, which makes it possible to be free of any risk of consecutive release of these nanoparticles in the state of aggregates.

The coating layer, precisely for this purpose, is formed from a material comprising at least one polymer A having a solubilization pH value within the pH range from 5 to 7 combined with at least one hydrophobic compound B and in particular as defined hereafter.

As is shown by what follows, this efficacy is reinforced by adjusting the thickness of the coating layer formed.

Microparticles

The reservoir-type microparticles according to the present invention are constituted by a core containing the active ingredient in a form combined with nanoparticles of at least one polymer POM, and by a coating surrounding the core.

The controlled release of the nanoparticles from the microparticles is ensured by the coating surrounding the core of each reservoir particle. This coating is designed in order to release the active ingredient and the polymer POM at very specific sites of the gastro-intestinal tract corresponding for example to the absorption windows of the active ingredient in the gastro-intestinal tract.

Due to the nature of this coating, the oral form considered according to the present invention can advantageously have a double release mechanism as a function of time and pH.

By this expression is meant that the oral form considered according to the invention has the following two specificities. Below the solubilization pH value of the polymer A forming the coating of its microparticles, the oral form according to the invention releases only a very limited quantity of nanoparticles. On the other hand, when it is present in the intestine or a comparable medium, it ensures an effective release of the nanoparticles. This release can then be carried out advantageously in less than 24 hours, in particular in less than 12 hours, in particular in less than 6 hours, in particular less than 2 hours or even in less than 1 hour.

In the case of the active ingredients having a very narrow absorption window, for example limited to the duodenum or the Peyer's patches, the release time of the nanoparticles is less than 2 hours and preferably less than 1 hour.

The size of the microparticles considered according to the invention is advantageously less than 2000 μm, in particular varies from 100 to 1000 μm, in particular from 100 to 800 μm and in particular from 100 to 500 μm.

Within the meaning of the invention, the size of the particles is expressed as a volume mean diameter D_{4,3} measured by laser granulometry using a Mastersizer 2000 device from Malvern Instrument equipped with the Sirocco 2000 dry route module.

As regards their coating, it is formed from a composite material obtained by mixture of:
at least one compound A having a solubilization pH value comprised within the pH range from 5 to 7;
at least one hydrophobic compound B;
and optionally at least one plasticizer and/or other conventional excipients.

Polymer A

Within the meaning of the present invention, the solubilization pH value of the polymer A is a pH value of the physiological medium or of the model in vitro medium below which the polymer is found in an insoluble state and above which this same polymer A is found in a soluble state.

For obvious reasons, this pH value is specific to a given polymer and directly linked to its intrinsic physico-chemical characteristics, such as its chemical nature and its chain length.

By way of non-limitative illustration of the polymers A suitable for the invention, there can in particular be mentioned:
methacrylic acid and methyl methacrylate copolymer(s),
methacrylic acid and ethyl acrylate copolymer(s),
cellulose derivatives such as:
   cellulose acetate phthalate (CAP),
   cellulose acetate succinate (CASE),
   cellulose acetate trimellitate (CAT),
   hydroxypropylmethylcellulose phthalate (or hypromellose phthalate) (HPMCP),
   hydroxypropylmethylcellulose acetate succinate (or hypromellose acetate succinate) (HPMCAS),
shellac gum,
polyvinyl acetate phthalate (PVAP),
and mixtures thereof.

According to a preferred embodiment of the invention, this polymer A is chosen from methacrylic acid and methyl methacrylate copolymer(s), methacrylic acid and ethyl acrylate copolymer(s) and mixtures thereof.

As specified previously, the polymer A considered according to the invention has a different solubility profile depending on whether it comes into contact with a pH value above or below its solubilization pH value.

Within the meaning of the invention, the polymer A is generally insoluble at a pH value below its solubilization pH value and by contrast soluble at a pH value above its solubilization pH value.

For example, it can be a polymer the solubilization pH value of which is:
5.0, such as for example hydroxypropylmethylcellulose phthalate and in particular that marketed under the name HP-50 by Shin-Etsu,
5.5, such as for example hydroxypropylmethylcellulose phthalate and in particular that marketed under the name HP-55 by Shin-Etsu or methacrylic acid and ethyl acrylate copolymer 1:1 and in particular that marketed under the name Eudragit L100-55 by Evonik,
6.0 such as for example a methacrylic acid and methyl methacrylate copolymer 1:1 and in particular that marketed under the name Eudragit L100 by Evonik,
7.0 such as for example a methacrylic acid and methyl methacrylate copolymer 1:2 and in particular that marketed under the name Eudragit S100 by Evonik.

All of these polymers are soluble at a pH value above their solubilization pH.

The coating is advantageously composed of 25 to 90%, in particular 30 to 80%, in particular 35 to 70%, or even 40 to 60% by weight of polymer(s) A in relation to its total weight.

More preferably, the polymer A is a methacrylic acid and ethyl acrylate copolymer 1:1.

Hydrophobic Compound B

According to a first variant, compound B can be selected from the products crystallized in the solid state and having a melting temperature T_{fb}≧40° C., preferably T_{fb}≧50° C., and still more preferably 40° C.≦T_{fb}≦90° C.

More preferably, this compound is then chosen from the following group of products:
vegetable waxes alone or in mixture with each other, such as those marketed under the trademarks DYNASAN P60 and DYNASAN 116;
hydrogenated vegetable oils alone or in mixture with each other; preferably chosen from the group comprising: hydrogenated cotton seed oil, hydrogenated soya oil, hydrogenated palm oil and mixtures thereof;
mono and/or di and/or tri esters of glycerol and of at least one fatty acid, preferably behenic acid, alone or in mixture with each other;
and mixtures thereof.

According to this embodiment, the B/A weight ratio can vary between 0.2 and 1.5 and preferably between 0.45 and 1.

More preferably, compound B is hydrogenated cotton seed oil.

Microparticles formed from such a coating are in particular described in the document WO 03/30878.

According to a second variant, the compound B can be a polymer which is insoluble in the fluids of the alimentary canal.

This polymer which is insoluble in the fluids of the alimentary canal or also the gastro-intestinal fluids is more particularly selected from:
non-water-soluble cellulose derivatives,
non-water-soluble (meth)acrylic (co)polymer derivatives,
and mixtures thereof.

More preferably, it can be chosen from ethylcellulose, and/or derivatives, for example those marketed under the name Ethocel®, cellulose acetate butyrate, cellulose acetate, ammonio (meth)acrylate copolymers, ethyl acrylate, methyl methacrylate and trimethylammonio ethyl methacrylate copolymers of type “A” or of type “B” in particular those marketed under the names Eudragit® RL and Eudragit® RS, poly(meth)acrylic acid esters, in particular those marketed under the name Eudragit® NE and mixtures thereof.

Ethylcellulose, cellulose acetate butyrate and the ammonio (meth)acrylate copolymers in particular those marketed under the names Eudragit RS® and Eudragit RL® are quite particularly suitable for the invention.

The coating of the microparticles then contains 10% to 75%, and can preferably contain 15% to 60%, more preferably 20% to 55%, or even 25 to 55% by weight, and still more particularly 30 to 50% polymer(s) A relative to its total weight.

Advantageously, the coating can then be formed, according to this embodiment, from a mixture of the two categories of polymers A and B in a polymer(s) B/polymer(s) A weight ratio greater than 0.25, in particular greater than or equal to 0.3, in particular greater than or equal to 0.4, in particular greater than or equal to 0.5, or even greater than or equal to 0.75.

According to another embodiment variant, the polymer(s) A/polymer(s) B ratio is moreover less than 8, in particular less than 4, or even less than 2 and more particularly less than 1.5.

By way of examples representative of the polymer A and B mixtures which are quite particularly suitable for the invention, there can in particular be mentioned the mixtures of ethylcellulose, cellulose acetate butyrate or ammonio (meth)acrylate copolymer of type A or B with at least one methacrylic acid and ethyl acrylate copolymer or a methacrylic acid and methyl methacrylate copolymer or a mixture thereof.

Apart from the abovementioned two types of compounds A and B, the coating of the particles according to the invention can comprise at least one plasticizer.

Plasticizer

This plasticizer can in particular be chosen from:
glycerol and its esters, and preferably from the acetylated glycerides, glyceryl-mono-stearate, glyceryl-triacetate, glyceryl-tributyrate,
the phthalates, and preferably from dibutyl phthalate, diethyl phthalate, dimethyl phthalate, dioctyl phthalate,
the citrates, and preferably from acetyl tributyl citrate, acetyl triethyl citrate, tributyl citrate, triethyl citrate,
the sebacates, and preferably from diethyl sebacate, dibutyl sebacate,
the adipates,
the azelates,
the benzoates,
chlorobutanol,
the polyethylene glycols,
the vegetable oils,
the fumarates, preferably diethyl fumarate,
the malates, preferably diethyl malate,
the oxalates, preferably diethyl oxalate,
the succinates; preferably dibutyl succinate,
the butyrates,
the cetyl alcohol esters,
the malonates, preferably diethyl malonate,
castor oil,
and mixtures thereof.

In particular, the coating can comprise less than 30% by weight, preferably 1% to 25% by weight, and, still more preferably, 5% to 20% by weight plasticizer(s) relative to its total weight.

According to a particularly advantageous embodiment, the coating layer has an average thickness greater than or equal to 25 μm, preferably greater than or equal to 30 μm, or even greater than or equal to 35 μm.

Such a thickness of the coating layer of the microparticle oral form according to the invention advantageously allows complete release of the active ingredient that it contains in a medium with a pH greater than 5, representative of that of the intestine.

According to another particular embodiment, the coating layer has a thickness less than 200 μm, more particularly less than or equal to 100 μm.

In particular, for particles of a size varying from 500 to 700 μm, the coating layer advantageously has a thickness varying from 25 to 50 μm.

The formation of the microparticles according to the invention can be carried out by any conventional technique suitable for the formation of a reservoir capsule the core of which is formed wholly or partly by at least one active ingredient non-covalently combined with nanoparticles of polymer POM, in particular as defined hereafter and supported or not supported on a neutral substrate, if appropriate using one or more binding agents and with one or more conventional excipients.

Thus, according to an embodiment variant, the nanoparticles non-covalently combined with the active ingredient can be present in the microparticles in a supported form.

Without this being limitative, the core of the microparticles can for example contain, apart from the nanoparticles combined with the active ingredient and the conventional excipients, sucrose and/or dextrose and/or lactose, or also a microparticle of an inert substrate such as cellulose serving as a support for said nanoparticles.

Thus, in a first preferred embodiment of the invention, the core of the microparticles is a granule containing the POM, the active ingredient, one or more binding agents ensuring the cohesion of the granule and various excipients known to a person skilled in the art. A coating is then deposited on this granule by any technique known to a person skilled in the art, and advantageously by spray coating.

The composition by weight of a microparticle according to this embodiment is the following:
the content by weight of nanoparticles loaded with active ingredient in the core is comprised between 0.1 and 80%, preferably between 2 and 70% preferably also between 10 and 60%;
the content by weight of binding agent in the core is comprised between 0.5 and 40%, preferably between 2 and 25%;
the content by weight of the coating in the microparticle is comprised between 5 and 50%, preferably between 15 and 35%.

In a second preferred embodiment, the core of the microparticles according to the invention comprises a neutral core around which a layer is deposited containing the active ingredient, the POM nanoparticles, a binding agent ensuring the cohesion of this layer and optionally different excipients known to a person skilled in the art, for example sucrose, trehalose and mannitol. The neutral core can be a particle of cellulose or sugar or any inert organic or saline compound which lends itself to coating.

The composition by weight of a particle according to this embodiment is then the following:
the content by weight of nanoparticles loaded with active ingredient in the core is comprised between 0.1 and 80%, preferably between 2 and 70% preferably also between 10 and 60%;
the content by weight of neutral core in the core of the microparticles is comprised between 5 and 50%, preferably between 10 and 30%;
the content by weight of binding agent in the core of the microparticles is comprised between 0.5 and 40%, preferably between 2 and 25%;
the content by weight of the coating in the microparticle is comprised between 5 and 50%, preferably between 15 and 35%.

Preferably, the microparticles are formed by spraying the compounds A and B and if present the other ingredients amongst which the plasticizer(s) generally in the solute state. This solvent medium generally contains organic solvents mixed or not mixed with water. The coating thus formed proves homogeneous in terms of composition as opposed to a coating formed by a dispersion of these same polymers, in a mostly aqueous liquid

According to a preferred embodiment variant, the sprayed solution contains less than 40% by weight water, in particular less than 30% by weight water and more particularly less than 25% by weight water.

Nanoparticles

As is clear from the above, the active ingredient contained in the core of the microparticles, forming the oral particulate form according to the invention, is present there in a form at least in part non-covalently combined with nanoparticles of at least one polymer POM.

The terms “combination” or “combined” used to qualify the relationships between one or more active ingredients and the polymer POM, mean that the active ingredient or ingredients are combined with the polymer(s) POM in particular by non-covalent physical interactions, in particular hydrophobic interactions, and/or electrostatic interactions and/or hydrogen bonds and/or via a steric encapsulation by the polymers POM.

This combination generally results from hydrophobic and/or electrostatic interactions and therefore assumes that the polymer POM incorporates in its structure units capable of producing this type of interaction.

These units, in particular hydrophobic or ionized, can be present directly within the hydrocarbon chain forming the skeleton of said polymer and/or can be formed by one or more hydrophobic or ionized groups borne by said hydrocarbon chain.

The expression “borne group” means that said group is pendant, i.e. that said group is a side group linked to the main chain of the polymer by one or more covalent bonds. For example, when the polymer is a polyamino acid comprising amino acid residues, said pendant group is a side group in relation to the amino acid residues and can be in particular a substituent of the carbonyl function in position y of the amino acid residue which bears it.

The polymers POM considered according to the invention generally have a degree of polymerization DP comprised between 10 and 1000, in particular between 30 and 500 and more particularly between 50 and 250 or even between 20 and 150.

The polymers POM considered according to the invention can moreover spontaneously form nanoparticles when they are dispersed in an aqueous medium and in particular water.

The nanoparticles can be anionic, cationic or neutral, and are preferably anionic or cationic.

Within the meaning of the present invention, by “anionic nanoparticles” is meant nanoparticles of a polymer POM the overall charge of which at a neutral pH is negative; and by “cationic nanoparticles” is meant nanoparticles of a polymer POM the overall charge of which at a neutral pH is positive.

The overall charge can be measured by any method known to a person skilled in the art, such as for example the measurement of the Zeta potential at a neutral pH.

In a general manner, the size of the nanoparticles varies from 1 to 1000 nm, in particular from 5 to 500 nm, in particular from 10 to 300 nm and more particularly from 10 to 100 nm. The size of the nanoparticles of POM is evaluated by the average hydrodynamic diameter of these particles. The measurement is carried out by quasi-elastic diffusion of light with a CGS-3 device from ALV. To this end, the suspension of POM is concentrated at 0.5 mg/ml in a saline medium such as 0.15 M NaCl after a rest period sufficient to achieve equilibrium.

Hydrocarbon Chain

As stated previously, the polymer POM according to the invention comprises a hydrophilic hydrocarbon chain bearing one or more hydrophobic groups (G) or an amphiphilic hydrocarbon chain.

According to a particular embodiment, it is a polymer comprising a hydrophilic hydrocarbon chain bearing one or more hydrophobic groups (G).

The hydrocarbon chain forming the polymer POM can be chosen from the polyamino acids, anionic polysaccharides such as dextran sulphate, carboxymethylcellulose, gum arabic, hyaluronic acid and its derivatives, the polygalacturonics, the polyglucuronics, or cationic polysaccharides, such as chitosan, or also collagen and its gelatin-type derivatives.

In view of the above, it is understood that within the meaning of the invention, the expression “hydrocarbon chain” covers the hydrocarbon chains which can contain one or more nitrogen atoms.

In what follows, the expressions “hydrocarbon chain” and “hydrocarbon chain which can contain one or more nitrogen atoms” are used interchangeably.

Advantageously, the hydrocarbon chain forming the polymer POM is a polyamino acid. According to an aspect of the invention, the polymer POM is biodegradable.

Within the meaning of the invention, the term “polyamino acid” covers both the natural polyamino acids and the synthetic polyamino acids, as well as the oligoamino acids comprising 10 to 20 amino acid residues in the same way as the polyamino acids comprising more than 20 amino acid residues.

The polyamino acids are synthetic linear polymers, advantageously composed of alpha-amino acids linked by peptide bonds.

There are numerous synthetic techniques for forming block or statistical polymers, multiple-chain polymers and polymers containing a determined amino acid sequence (cf. Encyclopedia of Polymer Science and Engineering, volume 12, page 786; John Wiley & Sons).

A person skilled in the art is capable, by virtue of their knowledge, of implementing these techniques in order to access polymers suitable for the invention. In particular, they can also refer to the teaching of the documents WO 96/29991, WO 03/104303, WO 96/079614 and PCT/EP/2008/055507. In the variant of the invention, where the hydrocarbon chain forming the polymer POM is amphiphilic in nature, this polyamino acid comprises at least one or even more neutral hydrophobic amino acids.

More particularly, such a polymer POM can be a polyamino acid comprising at least two types of recurrent amino acid residues AAN and AAI:
the type AAN corresponding to a neutral hydrophobic amino acid,
the type AAI corresponding to an amino acid with an ionizable side chain, at least some of the amino acids of type AAI being in the ionized form,
the amino acids of each type AAN and AAI being identical to or different from each other, and the molar mass by weight of said polyamino acid being greater than or equal to 2500 D, in particular greater than or equal to 4000 D, preferably greater than or equal to 5000 D.

Such polyamino acids are in particular described in the document WO 96/29991 the content of which is incorporated by way of reference.

In this embodiment variant of POM according to the invention, the AAN (or the AANs) is (are) more particularly chosen from the following list: Leu, Ile, Val, Ala, Pro, Phe and mixtures thereof and the AAI (or the AAIs) is (are) more particularly formed by the Glu and/or the Asp.

Still more preferably, such polyamino acids comprise a single type of AAI monomers corresponding, preferably, to Glu and a single type of AAN monomers corresponding, preferably, to Leu.

According to another embodiment variant, the hydrocarbon chain forming the polymer POM is a hydrophilic polyamino acid.

More particularly, the polyamino acids then forming such POMs are oligomers or homopolymers comprising recurrent glutamic or aspartic acid units or copolymers comprising a mixture of these two types of amino acid residues. The residues considered in these polymers preferably have the configuration D or L or D/L and are linked by their alpha or gamma positions in the case of the glutamate or glutamic acid residue and alpha or beta positions in the case of the aspartic acid or aspartate residue and more preferably have the configuration L and are linked by their alpha position.

Preferably, the polymer POM comprises a polyamino acid hydrocarbon chain formed by aspartic acid units and/or glutamic acid units, and at least some of these units bear grafts comprising at least one hydrophobic group (G).

According to an embodiment variant, the hydrocarbon chain is constituted by an alpha-L-glutamate or alpha-L-glutamic acid homopolymer.

According to another embodiment variant, the hydrocarbon chain is constituted by an alpha-L-aspartate or alpha-L-aspartic acid homopolymer.

According to another particularly preferred embodiment variant, the hydrocarbon chain is constituted by an alpha-L-aspartate/alpha-L-glutamate or alpha-L-aspartic/alpha-L glutamic acid copolymer.

Such polymers POM are in particular described in the documents WO 03/104303, WO 96/079614 and PCT/EP/2008/055507 the content of which is incorporated by way of reference. These polyamino acids can also be of the type of those described in the Patent Application PCT WO-A-00/30618.

These polymers can be obtained by methods known to a person skilled in the art.

A certain number of polymers which can be used according to the invention, for example, poly(alpha-L-glutamic acid), poly(alpha-D-glutamic acid), poly(alpha-D,L-glutamate) and poly(gamma-L-glutamic acid) type of variable masses are commercially available.

Poly(L-glutamic acid) can also be synthesized according to the route described in the Patent Application FR 2 801 226.

The polymerization chemistry and the coupling reactions of the groups are standard and well known to a person skilled in the art (see for example the applicant's abovementioned patents or patent applications).

More particularly, the polymer POM is a polyhydroxyalkylglutamine comprising a multiplicity of pendant hydrophobic groups (G), identical or different and preferably at least 2 hydrophobic groups (G) and if appropriate one or more cationic groups and/or one or more ionizable groups and/or one or more neutral groups.

In the present description, by “cationic group” is meant a group grafted covalently to a glutamic residue, and comprising one or more amine functions or one or more quaternary ammoniums. In the case of an amine function, the group is mainly ionized at any pH below its pKa, in the case of a quaternary ammonium, the group is ionized at any pH.

In the present description, by “neutral group” is meant a group bearing no charge in the case of any pH comprised between 3 and 10, for example the groups obtained by condensation with the carboxyl of a glutamic acid residue of ethanolamine (bound by nitrogen), amino-propane diol, an alkylene glycol or a polyoxyalkylene glycol.

A polymer POM, can in fact bear one or more grafts of polyalkylene glycol type bound to an amino acid unit constituting it. Preferably, the polyalkylene glycol is a polyethylene glycol and more particularly used with a molar percentage of grafting of polyethylene glycol varying from 1 to 30%.

It should moreover be noted that the residual carboxylic functions of the modified polyglutamate are either neutral (COOH form), or ionized (COO⁻ anion), depending on pH and composition. The following terms are therefore used interchangeably i) glutamate residue or glutamic acid residue, ii) polyglutamate or polyglutamic acid.

Hydrophobic Group

More particularly, the hydrophobic groups G are identical to or different from each other and are selected from the group comprising:
(i) the alkyls, the acyls or the linear or branched alkenyls, preferably linear C₁-C₂₀ and still more preferably C₂-C₁₈;
(ii) the hydrocarbon groups containing one or more heteroatoms, preferably those containing oxygen and/or sulphur and, still more preferably, those of the following formula:

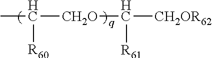

in which:
R₆₀ is a linear or branched alkyl, acyl or alkenyl, preferably linear C₁-C₂₀ and still more preferably C₂-C₁₈ group,
R₆₁ and R₆₂ are identical to or different from each other and correspond to hydrogen or to a linear or branched alkyl, acyl or alkenyl radical, preferably linear C₁-C₂₀ and still more preferably C₂-C₁₈ group,
q=1 to 100;
(iii) the aryls, the aralkyls or the alkylaryls, preferably the aryls;
(iv) the hydrophobic derivatives, preferably, the phosphatidylethanolamino-group or the groups chosen from octyloxy-, dodecyloxy-, tetradecyloxy-, hexadecyloxy-, octadecyloxy-, 9-octadecenyloxy-, tocopheryloxy- or cholesteryloxy-.

By “hydrocarbon groups”, is meant within the meaning of the present invention, groups comprising in particular hydrogen and carbon atoms.

Preferably, in this variant, the hydrophobic groups are selected from the following group: methyl, ethyl, propyl, docedyl, hexadecyl, octadecyl.

Particularly preferably, the hydrophobic groups (G) are chosen from the following group:
the linear or branched C₈ to C₃₀ alkyls which may optionally comprise at least one unsaturation and/or at least one heteroatom,
the C₈ to C₃₀ alkylaryls or arylalkyls which may optionally comprise at least one unsaturation and/or at least one heteroatom,
and the C₈ to C₃₀ (poly)cyclics which may optionally comprise at least one unsaturation and/or at least one heteroatom.

More precisely, at least one of the hydrophobic groups (G) is obtained by grafting, from a precursor chosen from the group comprising octanol, dodecanol, tetradecanol, hexadecanol, octadecanol, oleylalcool, tocopherol or cholesterol.

Advantageously, the hydrophobic groups G considered according to the invention comprise 8 to 30 carbon atoms.

According to a particular embodiment at least one and preferably all of the groups G present in a polymer POM form a tocopheryloxy-group.

Advantageously, at least one of the hydrophobic groups G is included in a hydrophobic graft comprising at least one spacer hinge (or unit) allowing linking of the hydrophobic G group to the structure of the polymer POM.

This hinge can comprise, e.g. at least one direct covalent bond and/or at least one amide bond and/or at least one ester bond. For example, the hinge can be of the type of those belonging to the group comprising in particular: the amino acid residues different from the monomeric unit constituting the hydrocarbon chain, the amino alcohol derivatives, the polyamine derivatives (for example the diamines), the polyol derivatives (for example the diols) and the hydroxyacid derivatives.

The grafting of the Gs to the amine chain can involve precursors of G, capable of binding to said chain.

The precursors of the Gs are, in practice and without this being limitative, chosen from the group comprising the alcohols and the amines, these compounds being able to be easily functionalized by a person skilled in the art.

The hinges forming hydrophobic grafts with the Gs can be di-, tri- or tetra-valent (or even pentavalent or more). In the case of a divalent hinge, the hydrophobic graft comprises a single group G, whereas a trivalent hinge confers a bifid character on the hydrophobic graft, i.e. the graft has two G substituents. As an example of a trivalent hinge there can be mentioned, inter alia, amino acid residues, for example glutamic acid or polyol residues, for example glycerol. Thus, two advantageous but non-limitative examples of hydrophobic grafts comprising bifid Gs are the dialkyl glycerols and the dialkyl glutamates. The coupling of the hydrophobic graft G is within the competence of a person skilled in the art and can in particular be carried out according to the protocol described in the documents PCT/EP2008/055507 and WO 03/104303.

Cationic or Neutral Group

The polyamino acid according to the invention can also bear cationic groups. These groups are grafted to the glutamic residues, preferably by means of an amide or ester bond.

According to another variant of the invention, the cationic groups can be chosen from those which comprise at least one quaternary ammonium or at least one strong base the semi-neutralization pH of which is greater than 8.0.

Such cationic groups can be obtained from the following precursor compounds:
a linear diamine with 2 to 6 carbon atoms, preferably putrescine,
agmatine,
ethanolamine bound by oxygen,
choline bound by oxygen,

an ester or amide derivative of an amino acid the side chain of which is positively charged at neutral pH, i.e. lysine, arginine, ornithine, bound by the amine function in the alpha position.

Thus, the cationic groups which can be used to functionalize the glutamate residues are identical to or different from each other and can correspond to:
a histidine derivative chosen from the group comprising the histidine esters, preferably methyl ester and ethyl ester, histidinol, histamine, histidinamide, the N-monomethyl derivative of histidinamide and the N,N′-dimethyl derivative of histidinamide
the following general formula:

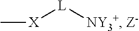

in which:

X═O, NH,

Y=independently an H or CH₃,

Z⁻=a chloride, sulphate, phosphate or acetate,

L=a linear (C₂ to C₆) alkylene optionally substituted by a functional group of carboxyl type or derivative.

More precisely, the cationic groups which can be used in the present invention are chosen from the following group:
—NH—(CH₂)_{w}—NH₃⁺, Z⁻ with w comprised between 2 and 6, and preferably w is equal to 4,
—O—(CH₂)₂—NH₃⁺, Z⁻,
—O—(CH₂)₂—N⁺(CH₃)₃, Z⁻,

a group chosen from the following group:
—NH—(CH₂)₄—NH—C(═NH)—NH₃⁺, Z⁻,
an amino acid residue or an amino acid derivative of formula:

in which:
R¹ is alkoxy, preferably —OMe or —OEt, or —R¹ is —NH₂, alkylamino, preferably —NH—CH₃ or —N(CH₃)₂;
R³ is —(CH₂)₄—NH₃⁺, Z⁻, —(CH₂)₃—NH—C(═NH)—NH₃⁺, Z⁻, —(CH₂)₃—NH₃⁺, Z⁻;

where Z⁻ is a chloride, a sulphate, a phosphate or an acetate, preferably a chloride.

For example, the cationic groups can have the following formulae:

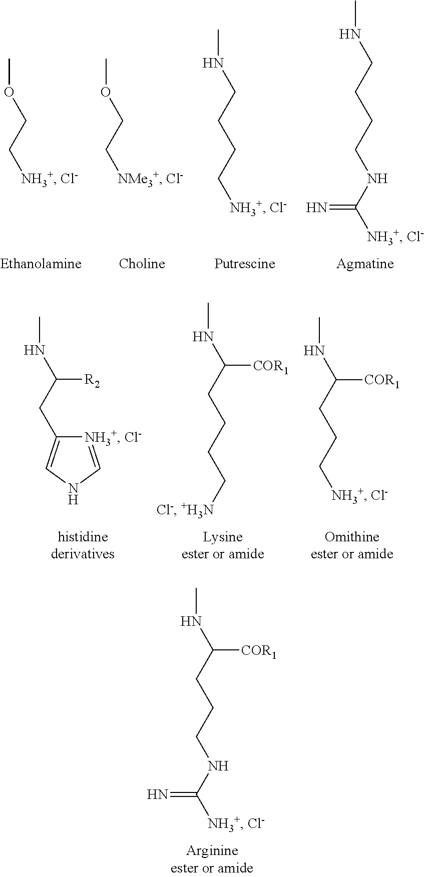

in which —R₁ represents an alkoxy or alkylamino, preferably an —OMe, —OEt, —NH₂, —NHCH₃ or —N(CH₃)₂, and —R₂ represents a hydrogen atom, —CH₂OH, CO₂H or —C(═O)—R₁.

The neutral groups can for their part be chosen from the following group: a hydroxyethylamino-, dihydroxypropylamino-, hydroxyalkyloxy- or polyoxyalkylene.

The coupling of the cationic and optionally neutral groups with an acid function of the polymer is carried out simultaneously in a second stage in the presence of a chloroformate as coupling agent and in an appropriate solvent such as dimethylformamide, N-methylpyrrolidone (NMP) or dimethylsulphoxide (DMSO).

In the case when the cationic group contains two chemically non-differentiated amine functions (e.g. linear diamine), it can be introduced in a form in which one of the two functions is protected. A last stage of cleavage of the protective group is then added.

The polymerization chemistry and the coupling reactions of the groups are standard and well known to a person skilled in the art (see for example the applicant's abovementioned patents or patent applications).

Polymer POM of General Formula I

According to a preferred varying from the invention, the polymer POM is a compound of the following formula (I) or one of its pharmaceutically acceptable salts,

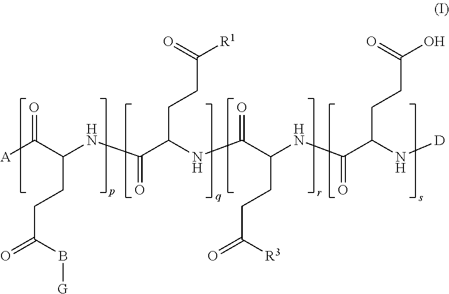

in which:
A represents independently:
   RNH— in which R represents an H, a linear C₂ to C₁₀ alkyl, a branched C₃ to C₁₀ alkyl or a benzyl,
   a terminal amino acid residue of formula:

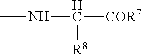

in which
—R⁷ is —OH, —OR⁹ or —NHR¹⁰, and
R⁸, R⁹ and R¹⁰ represent independently an H, a linear C₂ to C₁₀ alkyl, a branched C₃ to C₁₀ alkyl or a benzyl;
B is a direct bond, a group with a divalent, trivalent or tetravalent bond, preferably chosen from:
—O—, —NH—, —N(C₁₋₅ alkyl), an amino acid residue (preferably natural), diol, triol, diamine, triamine, aminoalcohol or hydroxyacid comprising 1 to 6 carbon atoms;
D represents an H, a linear C₂ to C₁₀ acyl, a branched C₃ to C₁₀ acyl, or a pyroglutamate;
the hydrophobic groups G each independently of each other are chosen from:
   the linear or branched C₈ to C₃₀ alkyls which can optionally comprise at least one unsaturation and/or at least one heteroatom (preferably O and/or N and/or S), or
   the C₈ to C₃₀ alkylaryls or arylalkyls which can optionally comprise at least one unsaturation and/or at least one heteroatom (preferably O and/or N and/or S), or
   the C₈ to C₃₀ (poly)cyclic groups which can optionally comprise at least one unsaturation and/or at least one heteroatom (preferably O and/or N and/or S);
R¹ is chosen from the following group:
   —NH—(CH₂)_{w}—NH₃⁺, Z⁻ with w comprised between 2 and 6, and preferably w is equal to 4,
   —NH—(CH₂)₄—NH—C(═NH)—NH₃⁺, Z⁻,
   —O—(CH₂)₂—NH₃⁺, Z⁻,
   —O—(CH₂)₂—N⁺(CH₃)₃, Z⁻,
   an amino acid residue or an amino acid derivative of formula:

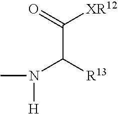

in which:
   X is an oxygen atom or an —NH—,
   R¹² is H, linear C₂ to C₁₀ alkyl, branched C₃ to C₁₀ alkyl or benzyl,
   —R³ is —(CH₂)₄—NH₃⁺, Z⁻, —(CH₂)₃—NH—C(═NH)—NH₃⁺, Z⁻, —(CH₂)₃—NH₃⁺, Z⁻¹;
with the counter-anion Z⁻ being a chloride, sulphate, phosphate or acetate, preferably a chloride;
   R³ represents a hydroxyethylamino-, dihydroxypropylamino, alkylene glycol residue, polyoxyalkylene glycol or a group of formula:

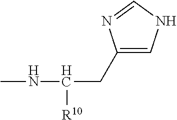

where —R¹⁰ represents —H, —CO₂H, an alkyl ester (preferably —COOMe or —COOEt), —CH₂OH, —C(═O)—NH₂, —C(═O)—NH—CH₃ or —C(═O)—N(CH₃)₂;
p, q, r and s are positive integers with q, r and s which may also be zero;
(p+q+r+s) which is the degree of polymerization DP varies from 10 to 1000, in particular from 20 to 500, preferably from 30 to 500;
the molar grafting rate of the hydrophobic groups G, (p)/(p+q+r+s) varies from 2 to 99 molar %, and preferably between 3 and 50% providing that each copolymer chain has at least 2 and preferably at least 3 hydrophobic groups;
the molar grafting rate of the cationic groups (q)/(p+q+r+s) varies from 0 to 98 molar %;
the molar grafting rate of the neutral groups (r)/(p+q+r+s), varies from 0 to 98 molar %;
the molar grafting rate of the anionic groups (s)/(p+q+r+s) varies from 0 to 98 molar %;
the overall charge level of the chain Q=(q−s)/(p+q+r+s) can be positive or negative;

the chain formation of the monomers of said general formula I being random, block, or multiblock type.

Such polymers are in particular described in detail in the document PCT/EP2008/055507 the content of which is incorporated by way of reference. For more details on their synthesis, reference to documents FR 02 07008 and FR 03 50190 will be helpful.

The general formula (I) described above must not be interpreted as representing only sequenced (or block) copolymers, but also random copolymers or multiblock copolymers.

By “pharmaceutically acceptable salts” of the polymer according to the invention is meant all of the polymers with the counter-ions combined with the ionized functions of the polymer. It can also be envisaged, for certain structures where there is co-existence of the positive and negative charges that there is a total or partial neutralization of the charges. A polymer having an equivalent number of positive charges and negative charges (isoelectric point) can exist without the presence of either counter-anion or counter-cation.

Preferably, the hydrophobic groups G, the anionic groups and the cationic groups are arranged in a random manner in pendant groups.

Preferably, the hydrophobic groups G are chosen from the following group: octyloxy-, dodecyloxy-, tetradecyloxy-, hexadecyloxy-, octadecyloxy-, 9-octadecenyloxy-, tocopheryloxy- or cholesteryloxy-, B then being a direct bond.

Quite particularly suitable for the invention are the compounds of general formula I′ corresponding to general formula I, in which:
A represents —NH₂
B is a direct bond,
D represents an H or a pyroglutamate;
the hydrophobic groups G each independently of each other are chosen from: octyloxy-, dodecyloxy-, tetradecyloxy-, hexadecyloxy-, octadecyloxy-, 9-octadecenyloxy-, tocopheryloxy- or cholesteryloxy- and
R³ represents a hydroxyethylamino-, or a dihydroxypropylamino.

The compounds of general formula I can be distinguished according to the chemical nature of the hydrophobic, cationic and/or anionic groups that they bear respectively and also as a function of the molar grafting rate in each of these groups.

Moreover, with regard to their percentage of grafting in cationic and/or anionic groups, the compounds of general formula I can be anionic, neutral or cationic at neutral pH.

Thus, according to a first embodiment variant the compounds are represented by a general formula I or I′ in which:
(p+q+r+s) varies from 20 to 250, and preferably from 50 to 225;
(p)/(p+q+r+s) varies preferably between 4 and 30% providing that each copolymer chain has at least 2 hydrophobic groups;
(q)/(p+q+r+s) is greater than or equal to 10%;
(r)/(p+q+r+s) is greater than or equal to 10%;
(s)/(p+q+r+s) is greater than or equal to 10%;
Q=(q−s)/(p+q+r+s), when it is positive, is comprised between +20% and +60% and, when it is negative, is less than −20%.

According to a second embodiment variant the compounds are represented by a general formula I or I′ in which
(p+q+r+s) varies from 20 to 250, and preferably from 50 to 225;
(p)/(p+q+r+s) varies preferably between 4 and 30% providing that each copolymer chain has at least 2 hydrophobic groups;
(q)/(p+q+r+s) is comprised between 10 and 80%, and preferably between 10 and 60%;
(r)/(p+q+r+s) is greater than or equal to 10%;
(s)/(p+q+r+s) is less than 15%.

According to a third embodiment variant the compounds are represented by a general formula I or I′ in which
(p+q+r+s) varies from 20 to 250, and preferably from 50 to 225;
(p)/(p+q+r+s) varies preferably between 4 and 30% providing that each copolymer chain has at least 2 hydrophobic groups;
(q)/(p+q+r+s) is greater than or equal to 10%;
(r)/(p+q+r+s) is less than 5%;
(s)/(p+q+r+s) is greater than 10%;
U Q=(q−s)/(p+q+r+s) is when it is positive comprised between +20% and +60%; and when it is negative, less than −20%.

According to a fourth embodiment variant the compounds are represented by a general formula I or I′ in which
(p+q+r+s) varies from 20 to 250, and preferably from 50 to 225;
(p)/(p+q+r+s) varies preferably between 4 and 30% providing that each copolymer chain has at least 2 hydrophobic groups;
(q)/(p+q+r+s) is less than 1%
(r)/(p+q+r+s) is less than 1%.

According to a particular embodiment, polymers according to the second abovementioned embodiment variant, the DP of which is comprised between 70 and 130, the (p)/(p+q+r+s) ratio varies between 7 and 13%, the (q)/(p+q+r+s) ratio varies between 30 and 50%, the (r)/(p+q+r+s) ratio varies between 40 and 60%, and the (s)/(p+q+r+s) ratio is less than 1% are quite particularly suitable for the invention as polymers POM.

In particular a polyglutamate cationic grafted 10% to vitamin E, 40% arginine and 50% ethanolamine may be suitable.

According to another particular embodiment, polymers according to the abovementioned fourth embodiment variant, the (p)/(p+q+r+s) ratio of which varies between 15 and 25% and the DP of which is comprised between 150 and 250 or between 70 and 130 are quite particularly suitable for the invention, as polymers POM.
A particular example is a polyglutamate grafted 10% with vitamin E.

Combination of the POM with an Active Ingredient

The techniques of combining one or more active ingredients with the polymer POM according to the invention and more particularly with the modified polyamino acids according to the invention are similar to those described in particular in the patent U.S. Pat. No. 6,630,171.

The active ingredients such as proteins, peptides or small molecules can combine spontaneously with the polymer POM of polyamino acid type. By small molecule is meant organic molecules with a mass of less than 1000 Da.

This combination is purely physical and does not involve the creation of a covalent bond between the active ingredient and the polymer.

Without being bound by the theory, it can be assumed that this non-specific combination is achieved by hydrophobic and/or electrostatic interaction, by hydrogen bond between the polymer and the active ingredient and/or by steric encapsulation of the active ingredient by the polymer. It is to be noted that it is not necessary, and often even undesirable, to combine the active ingredient with the nanoparticles by specific receptors of a peptide nature or of antigen/antibody or also enzyme/substrate type.

No stage of chemical crosslinking of the particles obtained is provided. The absence of chemical crosslinking makes it possible to avoid the chemical degradation of the active ingredient during the stage of crosslinking of the particles containing the active ingredient. Such a chemical crosslinking is in fact generally carried out by activation of polymerizable entities and involves potentially denaturing agents such as UV radiation, or glutaraldehyde.

The combination of the active ingredient and the polymer POM can in particular be carried out according to the following embodiments.

In a first embodiment, the active ingredient is dissolved in an aqueous solution and mixed with an aqueous suspension of the polymer POM.

In a second embodiment, the active ingredient in the form of powder is dispersed in an aqueous suspension of the polymer POM and the mixture is stirred until a homogeneous limpid suspension is obtained.

In a third embodiment, the polymer POM is introduced in the form of powder into an aqueous solution of the active ingredient.

In a fourth embodiment, the active ingredient and/or the polymer is dissolved in a solution containing an organic solvent miscible with water such as ethanol or isopropanol. The procedure according to embodiments 1 to 3 above is then followed. Optionally, this solvent can be eliminated by dialysis or any other technique known to a person skilled in the art.

For all of these embodiments, it can be advantageous to facilitate the interaction between the active ingredient and the polymer POM using ultrasound or a rise in temperature.

In the case when it is desirable to apply the AI/POM mixture to a neutral substrate of neutral sphere type, the following procedure can be followed:

A conventional binding agent intended to ensure the cohesion of the layer deposited on the neutral core is added to the homogeneous mixture of active ingredient and POM. Such binding agents are in particular proposed in Khankari R. K. et al., Binders and Solvents in Handbook of Pharmaceutical Granulation Technology, Dilip M. Parikh ed., Marcel Dekker Inc., New York, 1997.

The following are quite particularly suitable for the invention as binding agents: hydroxypropylcellulose (HPC), polyvinylpyrrolidone (PVP), methylcellulose (MC) and hydroxypropylmethylcellulose (HPMC).

The deposition of the corresponding mixture is then carried out by the standard techniques known to a person skilled in the art. This may in particular involve spraying the colloidal suspension of the nanoparticles loaded with active ingredients, and containing the binding agent and optionally other compounds, onto the support in a fluidized bed. For obvious reasons, the active ingredient/polymer POM weight ratio may vary significantly as a function of the dose of active ingredient considered.

More particularly, this ratio may vary between 0.1 and 300% by weight; between 1 and 100% by weight or between 5 and 80% by weight.

Active Ingredient

The active ingredients considered according to the invention are advantageously biologically active compounds which can be administered to an animal or human organism by oral route.

As examples of active ingredients which may combine with the polyamino acids according to the invention, there may be mentioned by way of non-limitative illustration:
the proteins such as insulin, the interferons, the growth hormones, the interleukins, erythropoietin or the cytokines;
the glycoproteins,
the proteins linked to one or more polyalkyleneglycol chains [preferably polyethyleneglycol (PEG): “PEGylated proteins”],
the peptides,
the polysaccharides,
the liposaccharides,
the oligonucleotides, the polynucleotides
and mixtures thereof.

In a general manner, they may be any therapeutic or cosmetic active ingredient, and therefore active ingredients other that those mentioned previously. Within the meaning of the invention the particulate oral forms dedicated to active pharmaceutical applications concern both human and veterinary therapy.

Preferably, the active ingredient is chosen from the group comprising the proteins or the peptides.

According to a particularly preferred embodiment, the active ingredient is insulin.

The present invention also relates to novel pharmaceutical or dietetic preparations developed from the microparticle oral form according to the invention.

This particulate form can thus be presented in the form of a powder, a suspension, a tablet or a gelatin capsule.

According to an embodiment variant, an oral form can comprise at least two types of nanoparticles, differing by the nature of the active ingredient and/or of the POM combined with said active ingredients.

According to yet another variant, which may be combined with the previous variant, an oral form can combine at least two types of microparticles differing from each other by the nature of their coating layer and/or of the active ingredient that they incorporate.

Finally, the invention also relates to a therapeutic treatment method consisting of ingestion according to a determined therapeutic dose, of a medicament comprising the microcapsules as defined above.

The invention is better explained by the examples hereafter, given only by way of illustration.

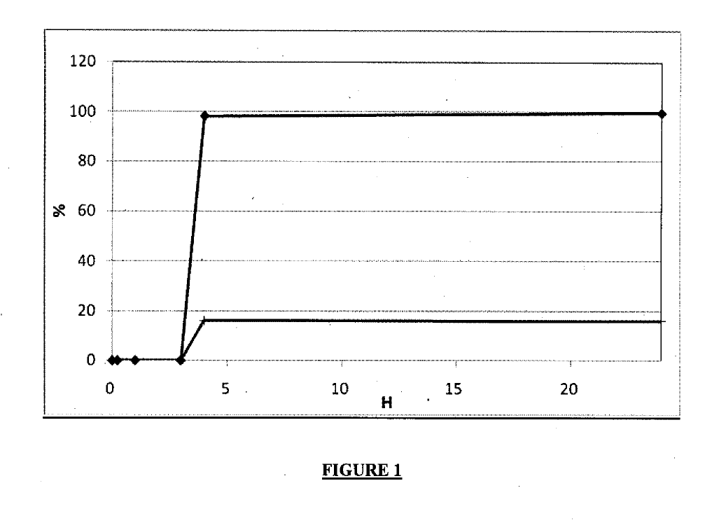

FIG. 1 represents the in vitro release profiles of carvedilol from the microparticles of Example 1, on the one hand of the free carvedilol not combined with the polymer POM (+) and, on the other hand, of the total carvedilol (♦), i.e. the free carvedilol and the carvedilol combined with the POM, in 0.1 N HCl medium over 3 hours then, after adjustment of the pH and salinity of the medium by the addition of 5 N soda and potassium phosphate, in 0.05 M medium at pH=7.0 as a function of time T in hours;

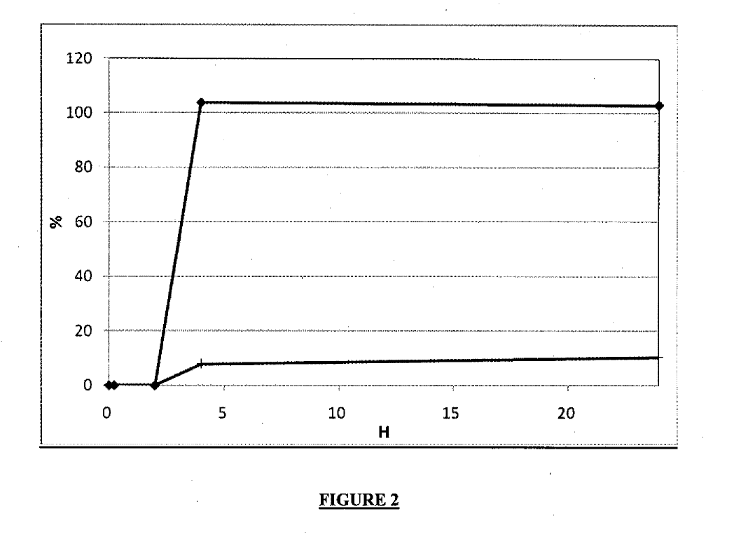

FIG. 2 represents the in vitro release profiles of insulin from the microparticles of Example 3, on the one hand, of the free insulin not combined with the POM (+) and, on the other hand, of the total insulin (♦), i.e. free and combined with the POM, in 0.1 N HCl medium over 3 hours then, after adjustment of the pH and salinity of the medium by the addition of 5 N soda and potassium phosphate, in 0.05 M medium at pH=7.0 as a function of time T in hours.

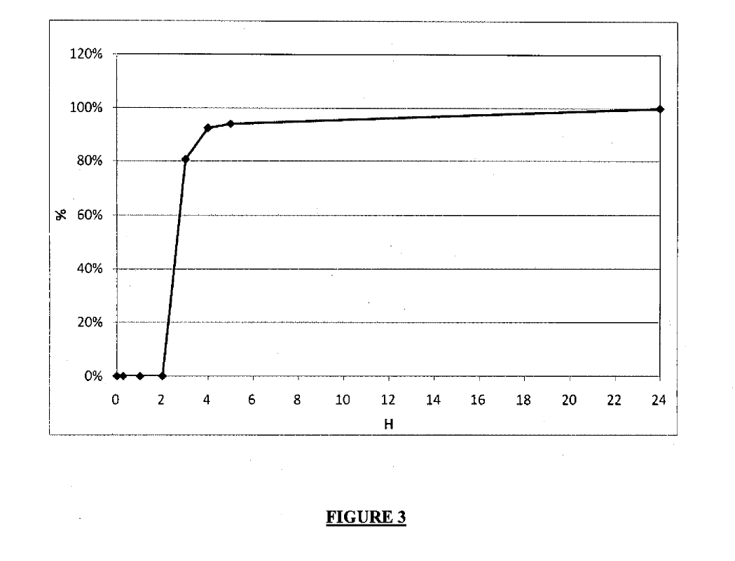

FIG. 3 represents the in vitro release profiles of insulin from the microparticles of Example 5, in 0.1 N HCl medium over 2 hours then, after adjustment of the pH and salinity of the medium by the addition of 5 N soda and potassium phosphate, in 0.05 M medium at pH=6.8 as a function of time T in hours.

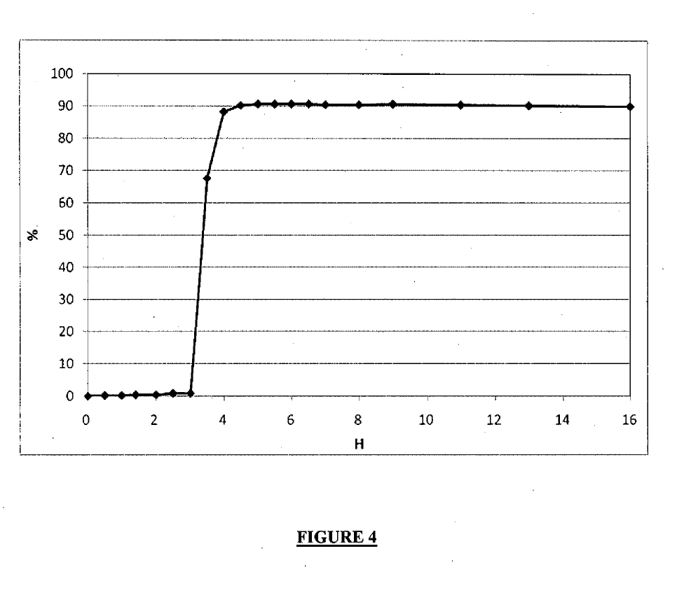

FIG. 4 represents the in vitro release profiles of carvedilol from the microparticles of Example 7, in 0.1 N HCl medium over 3 hours then, after adjustment of the pH and salinity of the medium by the addition of 5 N soda and potassium phosphate, in 0.05 M medium at pH=6.8 as a function of time T in hours.

### EXAMPLE 1

### Preparation and Formulation of Microparticles of Carvedilol Base Combined with a Polyglutamate Grafted 20% with Vitamin E and with a Degree of Polymerization of Approximately 100

Stage 1: Preparation of the Combination of Carvedilol Base with the Polyglutamate Polymer Grafted 20% with Vitamin E and with a Degree of Polymerization of Approximately 100 (pGlu-VE 100-20)

With reference to formula I, this polymer POM is characterized by: p+q+r+s=100, p=20, q=0, r=0, and s=80.

1.21 g of carvedilol base are introduced into a 250 ml glass flask. 133.29 g of an aqueous solution of pGlu-VE 100-20, at pH=7.0 and concentrated to 90 mg/g, are added. The preparation is placed in an ultrasound bath at ambient temperature until complete dissolution of the carvedilol base (i.e. until disappearance of non-solubilized carvedilol base powder). After dissolution of the carvedilol base, a perfectly limpid solution is obtained.

Stage 2: Preparation of Granules (Coating Stage)

12.5 g of sucrose (Compressuc PS from Tereos) and 6.3 g of povidone (Plasdone K29/32 from ISP) are introduced under magnetic stirring into a 250 ml glass flask containing 134.5 g of solution of carvedilol base combined with the pGlu-VE 100-20 prepared in Stage 1. Once the sucrose crystals and the povidone powder have dissolved, the solution is sprayed onto 38.0 g of cellulose spheres (Asahi Kasei) in a MiniGlatt fluidized bed in a bottom spray configuration (spraying of the coating solution via a nozzle situated in the bottom part of the bed of particles). After spraying, the product obtained is sieved on a 630 μm sieve. 70.5 g of granules, with a size of less than 630 μm, are then recovered.

Their volume mean diameter, determined by laser diffraction using a Mastersizer 2000 apparatus from Malvern Instruments equipped with the Sirocco 2000 dry route module is 536 μm.

580 mg of granules are introduced into a beaker containing 100 ml of 0.05 M potassium phosphate medium at pH=7.0 in order to obtain a polymer POM concentration in the suspension equal to approximately 1 mg/ml. The suspension is stirred by a magnetic bar for 2 hours at ambient temperature. 10 ml of the suspension are then removed and filtered on Acrodisc filters with a pore size of 0.45 μm. The hydrodynamic diameter of the nanoparticles then in suspension in the filtrate, determined in intensity mode by diffusion of light at an angle fixed at 90° using a CGS-3 device from ALV, is 14 nm.

Stage 3: Coating Phase

45.00 g of granules, as prepared in stage 2, are coated in a MiniGlatt fluidized bed, with 9.00 g of a methacrylic acid and ethyl acrylate copolymer (Eudragit L100-55 from Evonik) and 6.00 g of hydrogenated cotton seed oil (Lubritab from JRS Pharma) dissolved in 135.3 g of isopropanol at 78° C. After spraying, 57.90 g of microparticles are obtained. Their volume mean diameter, determined by laser diffraction using a Mastersizer 2000 apparatus from Malvern Instruments equipped with the Sirocco 2000 dry mode module, is 600 μm.

Thus, the average thickness of the coating deposited on the granule prepared during stage 2, calculated from the volume mean diameters determined for the granules obtained above in stage 2 and the microparticles obtained in stage 3, is 32 μm.

### EXAMPLE 2

### In Vitro Dissolution Tests

The in vitro release kinetics of the microparticles prepared in Example 1 is evaluated at 37° C.±0.5° C. in 900 ml of an 0.1 N HCl medium over 3 hours then, after adjustment of the pH and salinity of the medium by the addition of 5 N soda and potassium phosphate, in 900 ml of a 0.05 M medium at pH=7.0. The dissolution tests are carried out in a USP type II paddle device. The speed of rotation of the paddles is 100 rpm.

More precisely, the quantities present in the dissolution medium of free carvedilol, i.e. not combined with the pGlu-VE 100-20, on the one hand, and of total carvedilol, i.e. the free part and the part combined with the pGlu-VE 100-20, on the other hand, are monitored over time by HPLC liquid chromatography. For this purpose, at each sampling time, the samples of the dissolution medium are, on the one hand, analyzed directly by HPLC liquid chromatography in order to determine the proportion of total carvedilol, and, on the other hand, treated by ultrafiltration before analysis of the filtrate by HPLC in order to determine the proportion of carvedilol free base.

The test results are illustrated in FIG. 1.

It will be noted from FIG. 1 and Table I below, that most of the carvedilol released in the dissolution medium after adjustment of the pH and salinity of the medium is combined with the pGlu-VE 100-20.

TABLE I

| Hours | Total carvedilol (%) | Free carvedilol (%) |
|---|---|---|
| 0 | 0 | 0 |
| 3 | 0 | 0 |
| 4 | 98 | 16 |
| 24 | 100 | 16 |

### EXAMPLE 3

### Preparation and Formulation of Microparticles of Insulin Combined with a pGlu-VE Polymer

Stage 1: Preparation of the Combination of Insulin with the Polyglutamate Polymer Grafted 20% with Vitamin E and with a Degree of Polymerization of Approximately 100 (pGlu-VE 100-20)

2.40 g of insulin (Biocon) are introduced into a 250 ml glass flask. 133.7 g of aqueous solution of pGlu-VE 100-20, concentrated to 90 mg/g, are added. The preparation is placed in an ultrasound bath at ambient temperature until the insulin is completely dissolved. After dissolution of the insulin, a perfectly limpid solution is obtained.

Stage 2: Preparation of Granules (Coating Stage)

12.00 g of sucrose (Compressuc PS from Tereos) and 6.60 g of povidone (Plasdone K29/32 from ISP) are introduced under magnetic stirring into the 250 ml glass flask containing 136.1 g of insulin solution combined with the pGlu-VE 100-20, prepared previously. Once the sucrose crystals and povidone powder have dissolved, the solution is sprayed onto 38.00 g of cellulose spheres (from Asahi Kasei) in a fluidized bed in a bottom spray configuration (spraying of the coating solution through a nozzle situated in the bottom part of the bed of particles). After spraying, the product obtained is sieved on a 630 μm sieve. 66.2 g of granules, with a size of less than 630 μm, are then recovered.

Their volume mean diameter, determined by laser diffraction using a Mastersizer 2000 apparatus from Malvern Instruments equipped with the Sirocco 2000 dry route module is 535 μm.

580 mg of granules are introduced into a beaker containing 100 ml of 0.05 M potassium phosphate medium at pH=7.0 in order to obtain a polymer POM concentration in the suspension equal to approximately 1 mg/ml. The suspension is stirred with a magnetic bar for 2 hours at ambient temperature. 10 ml of the suspension are then removed and filtered on Acrodisc filters with a pore size of 0.45 μm. The hydrodynamic diameter of the nanoparticles, determined in intensity mode by diffusion of light at an angle fixed at 90° using a CGS-3 device from Malvern Instruments, is 12 nm.

Stage 3: Coating Phase

36.06 g of granules, as prepared above, are coated in a MiniGlatt fluidized bed, with 7.20 g of a methacrylic acid and ethyl acrylate copolymer (Eudragit L100-55 from Evonik) and 4.80 g of hydrogenated cotton seed oil (Lubritab from JRS Pharma), dissolved in 108.34 g of isopropanol at 78° C. After spraying, 46.30 g of microparticles are obtained. Their volume mean diameter, determined by laser diffraction using a Mastersizer 2000 apparatus from Malvern Instruments equipped with the Sirocco 2000 dry mode module is 623 μm.

Thus the average thickness of the coating deposited on the granule prepared during stage 2, calculated from the volume mean diameters determined for the granules obtained above in stage 2 and the microparticles obtained in stage 3, is 44 μm.

### EXAMPLE 4

### In Vitro Dissolution Tests

The in vitro release kinetics of the microparticles prepared in Example 3 is monitored at 37° C.±0.5° C. in 900 ml of a 0.1 N HCl medium over 3 hours then, after adjustment of the pH and salinity of the medium by the addition of 5 N soda and potassium phosphate, in 900 ml of a 0.05 M medium with pH=7.0. The dissolution tests are carried out in a USP type II paddle device. The speed of rotation of the paddles is 100 rpm.

More precisely, the quantities present in the dissolution medium of free insulin, i.e. not combined with the polymer pGlu-VE 100-20, on the one hand, and total insulin, i.e. the free part and the part combined with the polymer pGlu-VE 100-20, on the other hand, are monitored over time by HPLC liquid chromatography. For this purpose, each time a sample is taken, the samples of the dissolution medium are, on the one hand, analyzed directly by HPLC liquid chromatography in order to determine the proportion of total insulin and, on the other hand, treated by ultrafiltration before analysis of the filtrate by HPLC in order to determine the proportion of free insulin.

The test results are illustrated in FIG. 2.

It is noted that most of the insulin released, according to FIG. 2 and Table II below, in the dissolution medium after adjustment of the pH and salinity of the medium is combined with the polymer pGlu-VE 100-20.

TABLE II

| Hours | Total insulin (%) | Free insulin (%) |
|---|---|---|
| 0 | 0 | 0 |
| 2 | 0 | 0 |
| 4 | 103* | 8 |
| 24 | 103* | 10 |

*In accordance with acceptable experimental error limits

### EXAMPLE 5

### Preparation of Microparticles of Insulin Combined with a pGlu-VE-ARG-EA Cationic Polymer

Stage 1: Preparation of the Combination of Insulin with the Cationic Polyglutamate Polymer Grafted 10% with Vitamin E, 40% Arginine and 50% Ethanolamine

With reference to formula I, this polymer POM is characterized by: p+q+r+s=100, p=10, q=40, r=50, and s=0.

0.604 g of insulin (Biocon) are introduced into a 250 ml glass flask. 133.3 g of aqueous solution of polyglutamate polymer grafted 10% with vitamin E, 40% with arginine and 50% with ethanolamine, at pH 5.9 and concentrated to 79.4 mg/g, are added. The preparation is placed in an ultrasound bath at ambient temperature until complete dissolution of the insulin (i.e. until disappearance of non-solubilized insulin powder). After dissolution of the insulin, a perfectly limpid solution is obtained.

Stage 2: Preparation of Granules (Coating Stage)

6.0 g of sucrose (Compressuc PS from Tereos) and 4.4 g of povidone (Plasdone K29/32 from ISP) are introduced under magnetic stirring into the 250 ml glass flask containing 151.18 g of insulin solution combined with the polyglutamate grafted 10% with vitamin E, 40% with arginine and 50% with ethanolamine, prepared previously. Once the sucrose crystals and povidone powder have dissolved, the solution is sprayed onto 33.00 g of cellulose spheres (from Asahi Kasei) in a MiniGlatt fluidized bed in a bottom spray configuration (spraying of the coating solution through a nozzle situated in the bottom part of the bed of particles). After spraying, the product obtained is sieved on a 710 μm sieve. 37.4 g of granules, with a size of less than 710 μm, are then recovered. Their volume mean diameter, determined in intensity mode by laser diffraction using a Mastersizer 2000 apparatus from Malvern Instruments equipped with the Sirocco 2000 dry route module is 531 μm.

95.8 mg of granules are introduced into a beaker containing 20 ml of 0.05M phosphate medium at pH 6.8, in order to obtain a polymer POM concentration in the suspension equal to approximately 1 mg/ml. The suspension is stirred by a magnetic bar for 2 hours at ambient temperature. The suspension is then removed and filtered on Acrodisc filters with a pore size of 0.45 μm. The hydrodynamic radius of the nanoparticles then in suspension in the filtrate, determined in intensity mode by diffusion of light at an angle fixed at 90° using a CGS-3 device from Malvern Instruments, is 6 nm.

It should be noted that the hydrodynamic radius of the nanoparticles of polyglutamate grafted 10% with vitamin E, 40% with arginine and 50% with ethanolamine, before combination with insulin and determined by diffusion of light at a angle fixed at 90° using a CGS-3 device from Malvern Instruments, is 7 nm. The concentration of the solution was adjusted to 1 mg/ml in POM polymer before the measurement.

Stage 3: Coating Phase

30.0 g of granules, as prepared above, are coated in a MiniGlatt fluidized bed, with 2.0 g of a methacrylic acid and ethyl acrylate copolymer (Eudragit L100-55 from Evonik), 4.0 g of a methacrylic acid and methyl methacrylate copolymer (Eudragit S100 from Evonik) and 4.0 g of hydrogenated cotton seed oil (Lubritab from JRS Pharma), dissolved in 90.47 g of isopropanol at 78° C. After spraying, 39.7 g of microparticles are obtained. Their volume mean diameter, determined by laser diffraction using a Mastersizer 2000 apparatus from Malvern Instruments equipped with the Sirocco 2000 dry route module is 588 μm.

Thus the average thickness of the coating deposited on the granule prepared during stage 2, calculated from the volume mean diameters determined for the granules obtained above in stage 2 and the microparticles obtained in stage 3, is 28.5 μm.

### EXAMPLE 6

### In Vitro Dissolution Tests

The in vitro release kinetics of the microparticles prepared in Example 5 is monitored at 37° C.±0.5° C. in 500 ml of a 0.1 N HCl medium over 2 hours then, after adjustment of the pH and salinity of the medium by the addition of 5 N soda and potassium phosphate, in 500 ml of a 0.05 M medium at pH 6.8. Each of the samples of the dissolution medium is analyzed directly by HPLC chromatography in order to determine the proportion of insulin dissolved in the dissolution medium. The dissolution tests are carried out in a USP type II paddle device. The speed of rotation of the paddles is 100 rpm.

The test results are illustrated in FIG. 3.

It should be noted that the complete dose of insulin is released in the dissolution medium after adjustment of the pH and of the salinity of the medium.

### EXAMPLE 7

### Preparation of Microparticles of Carvedilol Base Combined with a pGlu-VE Polymer

Stage 1: Preparation of the Combination of Carvedilol Base with the pGlu-VE Polymer Grafted 10% with Vitamin E

With reference to formula I, this polymer POM is characterized by: p+q+r+s=100, p=10, q=0, r=0, and s=90.

1.01 g of carvedilol base are introduced into a 250 ml glass flask. 151.2 g of aqueous solution of polyglutamate polymer grafted 10% with vitamin E, at pH 6.9 and concentrated to 52.8 mg/g, are added. The preparation is placed in an ultrasound bath at ambient temperature until complete dissolution of the carvedilol base (i.e. until disappearance of non-solubilized carvedilol base powder). After dissolution of the carvedilol base, a perfectly limpid solution is obtained.

Stage 2: Preparation of Granules (Coating Stage)

4.00 g of sucrose (Compressuc PS from Tereos) and 3.03 g of povidone (Plasdone K29/32 from ISP) are introduced under magnetic stirring into the 250 ml glass flask containing 152.2 g of carvedilol base solution combined with the polyglutamate grafted 10% with vitamin E, prepared previously. Once the sucrose crystals and povidone powder have dissolved, the solution is sprayed onto 30.00 g of cellulose spheres (from Asahi Kasei) in a MiniGlatt fluidized bed in a bottom spray configuration (spraying of the coating solution through a nozzle situated in the bottom part of the bed of particles). After spraying, the product obtained is sieved on a 630 μm sieve. 46.0 g of granules, with a size of less than 630 μm, are then recovered. Their volume mean diameter, determined by laser diffraction using a Mastersizer 2000 apparatus from Malvern Instruments equipped with the Sirocco 2000 dry route module is 497 μm.

300 mg of granules are introduced into a beaker containing 50 ml of 0.05M phosphate medium at pH 6.8, in order to obtain a polymer POM concentration in the suspension equal to approximately 1 mg/ml. The suspension is stirred by a magnetic bar for 2 hours at ambient temperature. 10 ml of the suspension are then removed and filtered on Acrodisc filters with a pore size of 0.45 μm. The hydrodynamic radius of the nanoparticles then in suspension in the filtrate, determined in intensity mode by diffusion of light at an angle fixed at 90° using a CGS-3 device from Malvern Instruments, is 18 nm.

Stage 3: Coating Phase

36.00 g of granules, as prepared above, are coated in a MiniGlatt fluidized bed, with 3.85 g of a methacrylic acid and ethyl acrylate copolymer (Eudragit L100-55 from Evonik), 2.17 g of a methacrylic acid and methyl methacrylate copolymer (Eudragit S100 from Evonik) and 6.00 g of hydrogenated cotton seed oil (Lubritab from JRS Pharma), dissolved in 108.78 g of isopropanol at 78° C. After spraying, 44.8 g of microparticles are obtained. Their volume mean diameter, determined by laser diffraction using a Mastersizer 2000 apparatus from Malvern Instruments equipped with the Sirocco 2000 dry route module is 571 μm.

Thus the average thickness of the coating deposited on the granule prepared during stage 2, calculated from the volume mean diameters determined for the granules obtained above in stage 2 and the microparticles obtained in stage 3, is 37 μm.

### EXAMPLE 8

### In Vitro Dissolution Tests

The in vitro release kinetics of the microparticles prepared in Example 7 is monitored at 37° C.±0.5° C. by UV spectrometry in 900 ml of 0.1 N HCl over 3 hours then, after adjustment of the pH and salinity of the medium, at pH 6.8 and 0.05 M potassium phosphate. The dissolution tests are carried out in a USP type II paddle device. The speed of rotation of the paddles is 100 rpm.

The profiles obtained are shown in FIG. 4.

It should be noted that the carvedilol base is released in its entirety in the dissolution medium after adjustment of the pH and salinity of the medium.

1. Microparticle oral form, useful for conditioning at least one active ingredient and releasing in vivo this active ingredient according to a release profile controlled as a function of the pH and/or of time, comprising at least microparticles having a core containing at least said active ingredient and coated with at least one coating layer influencing said release profile of said active ingredient characterized in that:
the coating layer is formed from a material comprising at least one polymer A having a solubilization pH value within the pH range from 5 to 7 combined with at least one hydrophobic compound B, and
said active ingredient, present in said core of the microparticles, is at least in part non-covalently combined with nanoparticles formed from at least one polymer POM, said polymer comprising a hydrophilic hydrocarbon chain bearing one or more hydrophobic groups (G) or an amphiphilic hydrocarbon chain.

2. Oral form according to claim 1, in which said polymer POM can form nanoparticles spontaneously when it is dispersed in an aqueous medium and in particular water.

3. Oral form according to claim 1 or 2, in which the nanoparticles non-covalently combined with said active ingredient are used in a supported form.

4. Oral form according to any one of the previous claims, in which the size of the microparticles is less than 2000 μm, in particular varies from 100 to 1000 μm, in particular from 100 to 800 μm and in particular less than 100 to 500 μm.

5. Oral form according to any one of the previous claims, in which the size of the nanoparticles varies from 1 to 1000 nm, in particular from 5 to 500 nm, in particular from 10 to 300 nm and more particularly from 10 to 100 nm.

6. Oral form according to any one of the previous claims, in which the coating layer has an average thickness greater than or equal to 25 μm, preferably greater than or equal to 30 μm, or even greater than or equal to 35 μm.

7. Oral form according to any one of the previous claims capable, when it is present in the intestine or a comparable medium, of releasing in less than 24 hours, in particular in less than 12 hours, in particular in less than 6 hours in particular less than 2 hours or even in less than 1 hour the nanoparticles that it contains.

8. Oral form according to any one of the previous claims, in which the hydrocarbon chain is chosen from the group consisting of the polyamino acids, anionic polysaccharides such as dextran sulphate, carboxymethylcellulose, gum arabic, hyaluronic acid and its derivatives, the polygalacturonics, the polyglucuronics, or cationic polysaccharides, such as chitosan, or also collagen and its gelatin-type derivatives.

9. Oral form according to any one of the previous claims, in which the hydrocarbon chain is formed by a linear polyamino acid, with α-peptide chain formation.

10. Oral form according to any one of the previous claims in which the polymer POM is a polyamino acid comprising at least two types of recurrent amino acids AAN and AAI:
the type AAN corresponding to a neutral hydrophobic amino acid,
the type AAI corresponding to an amino acid with an ionizable side chain, at least some of the recurrent amino acids of type AAI being in ionized form,
the recurrent amino acids of each type AAN and AAI being identical to or different from each other,
and the molar mass by weight of said polyamino acid being greater than or equal to 2500 D, in particular greater than or equal to 4000 D, preferably greater than or equal to 5000 D.

11. Oral form according to any one of claims 1 to 9 in which the polymer POM is a polyamino acid formed from aspartic acid and/or glutamic acid units, at least some of these units bearing grafts comprising at least one hydrophobic group (G).

12. Oral form according to any one of claims 1 to 9 and 11, in which the hydrocarbon chain is constituted by an alpha-L-glutamate or alpha-L-glutamic acid homopolymer.

13. Oral form according to any one of claims 1 to 9 and 11, in which the hydrocarbon chain is constituted by an alpha-L-aspartate or alpha-L-aspartic acid homopolymer.

14. Oral form according to any one of claims 1 to 9 and 11, in which the hydrocarbon chain is constituted by an alpha-L-aspartate/alpha-L-glutamate or alpha-L-aspartic/alpha-L glutamic acid copolymer.

15. Oral form according to any one of claims 1 to 9 and 11 or 12, in which the polymer POM is a polyhydroxyalkylglutamine comprising at least a multiplicity of pendant hydrophobic groups (G), which are identical or different.

16. Oral form according to any one of claims 1 to 9 and 11, 12 or 13, characterized in that it comprises as polymer POM at least one compound of the following formula (I) or one of its pharmaceutically acceptable salts, 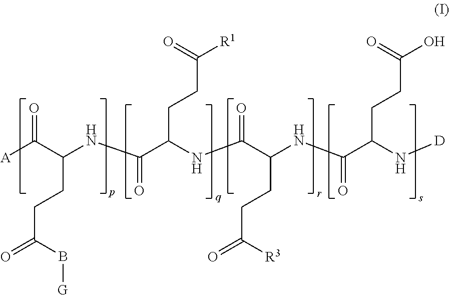
in which:
A represents independently:
RNH— in which R represents an H, a linear C₂ to C₁₀ alkyl, a branched C₃ to C₁₀ alkyl or a benzyl,
a terminal amino acid residue of formula: 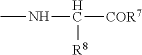
in which
—R⁷ is —OH, —OR⁹ or —NHR¹⁰, and
R⁸, R⁹ and R¹⁰ represent independently an H, a linear C₂ to C₁₀ alkyl, a branched C₃ to C₁₀ alkyl or a benzyl;
B is a direct bond, a group with a divalent, trivalent or tetravalent bond, preferably chosen from:
—O—, —NH—, —N(C₁₋₅ alkyl), an amino acid residue, diol, triol, diamine, triamine, amino alcohol or hydroxyacid comprising 1 to 6 carbon atoms;
D represents an H, a linear C₂ to C₁₀ acyl, a branched C₃ to C₁₀ acyl, or a pyroglutamate;
the hydrophobic groups G each independently of each other are chosen from:
the linear or branched C₈ to C₃₀ alkyls which can optionally comprise at least one unsaturation and/or at least one heteroatom (preferably O and/or N and/or S), or
the C₈ to C₃₀ alkylaryls or arylalkyls which can optionally comprise at least one unsaturation and/or at least one heteroatom (preferably O and/or N and/or S), or
the C₈ to C₃₀ (poly)cyclic groups which can optionally comprise at least one unsaturation and/or at least one heteroatom (preferably O and/or N and/or S);
and preferably are chosen from the following group: octyloxy-, dodecyloxy-, tetradecyloxy-, hexadecyloxy-, octadecyloxy-, 9-octadecenyloxy-, tocopheryloxy- or cholesteryloxy-, B then being a direct bond;
R¹ is chosen from the following group:
—NH—(CH₂)_{w}—NH₃⁺, Z⁻ with w comprised between 2 and 6, and preferably w is equal to 4,
—NH—(CH₂)₄—NH—C(═NH)—NH₃⁺, Z⁻,
—O—(CH₂)₂—NH₃⁺, Z⁻,
—O—(CH₂)₂—N⁺(CH₃)₃, Z⁻,
an amino acid residue or an amino acid derivative of formula: 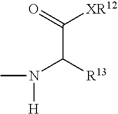
in which:
X is an oxygen atom or an —NH—,
R¹² is H, linear C₂ to C₁₀ alkyl, branched C₃ to C₁₀ alkyl or benzyl,
—R¹³ is —(CH₂)₄—NH₃⁺, Z⁻, —(CH₂)₃—NH—C(═NH)—NH₃⁺, Z⁻,
—(CH₂)₃—NH₃⁺, Z⁻;
in which the counter-anion Z⁻ is a chloride, sulphate, phosphate or acetate, preferably a chloride;
R³ represents a hydroxyethylamino-, a dihydroxypropylamino, an alkylene glycol residue, a polyoxyalkylene glycol or a group of formula: 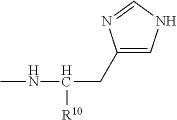
where —R¹⁰ represents —H, —CO₂H, an alkyl ester (preferably —COOMe or —COOEt), CH₂OH, —C(═O)—NH₂, —C(═O)—NH—CH₃ or —C(═O)—N(CH₃)₂;
a p, q, r and s are positive integers with q, r and s which may also be zero;
(p+q+r+s) varies from 10 to 1000, in particular from 20 to 500, and preferably from 30 to 500;
the molar grafting rate of the hydrophobic groups G, (p)/(p+q+r+s) varies from 2 to 99 molar %, and preferably between 3 and 50% providing that each copolymer chain has at least 2 and preferably at least 3 hydrophobic groups;
the molar grafting rate of the cationic groups (q)/(p+q+r+s) varies from 0 to 98 molar %;
the molar grafting rate of the neutral groups (r)/(p+q+r+s), varies from 0 to 98 molar %;
the molar grafting rate of the anionic groups (s)/(p+q+r+s) varies from 0 to 98 molar %;
the overall charge level of the chain Q=(q−s)/(p+q+r+s) can be positive or negative;
the chain formation of the monomers of said general formula I being random, monoblock or multiblock type.

17. Oral form according to the previous claim in which
A represents —NH₂
B is a direct bond,
D represents an H or a pyroglutamate;
the hydrophobic groups G each independently of each other are chosen from: octyloxy-, dodecyloxy-, tetradecyloxy-, hexadecyloxy, octadecyloxy-, 9-octadecenyloxy-, tocopheryloxy- or cholesteryloxy-, and
R³ represents a hydroxyethylamino-, or a dihydroxypropylamino.

18. Oral form according to claim 16 or 17 in which:
(p+q+r+s) varies from 20 to 250, and preferably from 50 to 225;
(p)/(p+q+r+s) varies preferably between 4 and 30% providing that each copolymer chain has at least 2 hydrophobic groups;
(q)/(p+q+r+s) is greater than or equal to 10%;
(r)/(p+q+r+s) is greater than or equal to 10%;
(s)/(p+q+r+s) is greater than or equal to 10%;
Q=(q−s)/(p+q+r+s) when it is positive, is comprised between +20% and +60% and when it is negative is less than −20%.

19. Oral form according to claim 16 or 17 in which
(p+q+r+s) varies from 20 to 250, and preferably from 50 to 225;
(p)/(p+q+r+s) varies preferably between 4 and 30% providing that each copolymer chain has at least 2 hydrophobic groups;
(q)/(p+q+r+s) is comprised between 10 and 80%, and preferably between 10 and 60%;
a (r)/(p+q+r+s) is greater than or equal to 10%;
(s)/(p+q+r+s) is less than 15%;

20. Oral form according to claim 16 or 17 in which
(p+q+r+s) varies from 20 to 250, and preferably from 50 to 225;
(p)/(p+q+r+s) varies preferably between 4 and 30% providing that each copolymer chain has at least 2 hydrophobic groups;
(q)/(p+q+r+s) is greater than or equal to 10%;
(r)/(p+q+r+s) is less than 5%;
(s)/(p+q+r+s) is greater than 10%;
Q=(q−s)/(p+q+r+s) when it is positive is comprised between +20% and +60%; and when it is negative is less than −20%.

21. Oral form according to claim 16 or 17 in which
(p+q+r+s) varies from 20 to 250, and preferably from 50 to 225;
(p)/(p+q+r+s) varies preferably between 4 and 30% providing that each copolymer chain has at least 2 hydrophobic groups;
(q)/(p+q+r+s) is less than 1% and
(r)/(p+q+r+s) is less than 1%.

22. Oral form according to claim 16 or 17 or 21, in which
the (p)/(p+q+r+s) ratio varies between 15 and 25%;
(q)/(p+q+r+s) is less than 1% and
(r)/(p+q+r+s) is less than 1%
and the degree of polymerization is, for example, comprised between 150 and 250 or 70 and 130.

23. Oral form according to any one of claims 1 to 9 and 11 to 22 in which at least one and preferably all of the groups G form a tocopheryloxy group.

24. Oral form according to any one of the previous claims, characterized in that the polymer POM has a degree of polymerization DP comprised between 10 and 1000, 30 and 500 and more particularly between 50 and 250.

25. Oral form according to any one of the previous claims, characterized in that the POM bears at least one graft of polyalkylene glycol type linked to a glutamate and/or aspartate unit.

26. Oral form according to the previous claim, in which the polyalkylene glycol is a polyethylene glycol and more particularly used with a molar percentage of grafting of polyethylene glycol varying from 1 to 30%.

27. Oral form according to any one of the previous claims, in which the polymer A is chosen from the methacrylic acid and methyl methacrylate copolymer(s), methacrylic acid and ethyl acrylate copolymer(s), cellulose derivatives such as cellulose acetate phthalate, cellulose acetate succinate, cellulose acetate trimellilate, hydroxypropylmethylcellulose phthalate, hydroxypropylmethylcellulose acetate succinate, shellac gum, polyvinyl acetate phthalate, and mixtures thereof.

28. Oral form according to any one of the previous claims, in which the coating of the microparticles contains 25 to 90% by weight, in particular 30% to 80% by weight, in particular 35% to 70% by weight, even 40 to 60% of polymer(s) A relative to its total weight.

29. Oral form according to any one of the previous claims, in which the hydrophobic compound B is selected from the crystallized products in the solid state, and having a melting temperature T_{fb}≧40° C., preferably T_{fb}≧50° C., and still more preferably 40° C.≦T_{fb}≦90° C.

30. Oral form according to the previous claim, in which compound B is chosen from the:
vegetable waxes;
hydrogenated vegetable oils alone or in mixture with each other; preferably chosen from the group comprising: hydrogenated cotton seed oil, hydrogenated soya oil, hydrogenated palm oil;
mono and/or di and/or tri esters of glycerol and of at least one fatty acid, preferably behenic acid, alone;
and mixtures thereof.

31. Oral form according to any one of claims 1 to 28, in which compound B is a polymer which is insoluble in the gastrointestinal fluids.

32. Oral form according to the previous claim in which said polymer B is chosen from:
the non-hydrosoluble cellulose derivatives and more particularly cellulose acetate butyrate, cellulose acetate,
the non-hydrosoluble derivatives of (meth)acrylic (co)polymers and more particularly ethyl acrylate, methyl methacrylate and trimethylammonio ethyl methacrylate copolymers of type “A” or of type “B”, and the poly(meth)acrylic acid esters.

33. Oral form according to any one of the previous claims, in which the active ingredient is a molecule of therapeutic or cosmetic interest.

34. Oral form according to any one of the previous claims, in which the active ingredient is a protein, a glycoprotein, a polysaccharide, a liposaccharide, an oligonucleotide, a polynucleotide or a peptide.

35. Oral form according to any one of the previous claims, in which the active ingredient is insulin.

36. Oral form according to any one of the previous claims comprising at least two types of nanoparticles, said nanoparticles differing by the nature of the active ingredient and/or of the POM combined with said active ingredients.

37. Oral form according to any one of the previous claims combining at least two types of microparticles differing from each other by the nature of their coating layer and/or of the active ingredient that they incorporate.

38. Oral form according to any one of the previous claims formulated in the state of a powder, a suspension, or in the form of a tablet or a gelatin capsule.

39. Oral form according to any one of the previous claims, characterized in that it is intended for the preparation of medicaments, and/or cosmetic products.

40. Oral form according to any one of the previous claims, suitable for releasing in a first phase the active ingredient combined with the nanoparticles of polymer(s) POM then in a second phase dissociating the active ingredient from said nanoparticles.

41. Method for the preparation of microparticles useful for conditioning at least one active ingredient and releasing in vivo this active ingredient according to a release profile controlled as a function of the pH and/or of time, said microparticles having a core containing at least said active ingredient and coated with at least one coating layer influencing said release profile of said active ingredient, said method comprising at least the stages consisting of:
a) having at least one active ingredient non-covalently combined with nanoparticles formed from at least one polymer POM comprising a hydrophilic hydrocarbon chain bearing one or more hydrophobic groups (G) or comprising an amphiphilic hydrocarbon chain,
b) forming from the nanoparticles of stage a) a core comprising said nanoparticles and one or more excipients,
c) forming from at least one polymer A having a solubilization pH value within the pH range from 5 to 7 and at least one hydrophobic compound B, a coating layer arranged around the core formed in stage b), and
d) recovering the expected microparticles.

42. Method according to the previous claim in which stage c) is carried out by spraying in fluidized bed on the nanoparticles of the stage b) at least one polymer A having a solubilization pH value within the pH range from 5 to 7 combined with at least one hydrophobic compound B.

43. Method according to claim 41 or 42 in which the particles of stage a) are as defined in claims 2 to 26.

44. Method according to any one of claims 40 to 43, in which the polymer A and the compound B are as defined in claims 27 to 32.
